(19) United States
(12) Patent Application Publication
(10) Pub. No.: US 2018/0289291 A1
(43) Pub. Date: Oct. 11, 2018

(54) HEALTH DIAGNOSTIC FINGERPRINT SCANNER

(71) Applicant: Bunnie Richie, Scottsdale, AZ (US)

(72) Inventor: Bunnie Richie, Scottsdale, AZ (US)

(21) Appl. No.: 15/482,420

(22) Filed: Apr. 7, 2017

(51) Int. Cl. A61B 5/1172; G06F 19/00; A61B 5/00

(52) U.S. Cl. CPC A61B 5/1172; G06F 19/321; A61B 5/0013; A61B 5/0022; A61B 5/0064; A61B 2576/02; A61B 5/441; A61B 5/7264; A61B 5/004; A61B 5/6898; G06F 19/345; A61B 5/0077

(57) ABSTRACT

Systems and methods allow for evaluating health conditions based on fingerprints and other skin patterns. A smartphone or other personal electronic device captures patient scans, such as fingerprint patterns transmitted from a wireless fingerprint scanner. Scans can be transmitted to a network-connected server for storage and analysis. The nature, frequency and location of patterns are compared to known correlations to generate a condition output. The condition output may then be made available to a patient and/or a medical professional, such as via notification message and/or access to a web portal.

### TECHNICAL FIELD

The present disclosure relates in general to health diagnostic devices, and in particular to a health diagnostic device for scanning portions of a patient's skin containing distinguishable patterns, such as finger tips.

### BACKGROUND

As the costs of traditional healthcare service rise, individuals and health care professionals increasingly seek to reduce the cost, and increase the efficacy, of diagnostic solutions. Meanwhile, we continually seek to improve access to health care services, particularly for underprivileged individuals and individuals living in remote areas. While recent advancements in many areas of healthcare technology, such as genetic testing, have made such tests more accessible than in the past, they still typically require access to expensive equipment and expert personnel. They may also require a long period of time to get results. Thus, the results of such tests may be inaccessible to many, or too delayed for optimal usefulness.

In view of these and other issues, it may be desirable to exploit opportunities to provide low cost health testing and evaluation, which may be performed using relatively inexpensive equipment.

### SUMMARY

Methods and apparatuses are described for evaluating a patient's health condition based on scans of a patient's skin prints. In some embodiments, a method and/or apparatus involves capturing a set of one or more images of a patient's skin print patterns by a skin print scanner and a personal electronic device. Computer-implemented analysis application logic may be executed to evaluate the set of images. A pattern recognition module may identify the presence or absence of predetermined patterns (e.g. whorls, arches, loops and ridges), at specific locations within a patient's scans and/or with particular frequency of occurrence. Based on the output of the pattern recognition application logic, a report may be generated (e.g. by an application logic evaluation module), indicative of a health condition of the patient.

In some embodiments, images may be captured by a fingerprint scanner device and transmitted to a smartphone located nearby. In some embodiments, the smartphone may then transmit the set of images to a network-connected server, such as a central cloud server, for application of pattern recognition operations and generation of a health condition report. Health condition reports may then be published to the patient and/or a patient representative, such as a doctor. Health condition reports may be published by operations including making the report available via a secure web site. In other embodiments, scan images may be processed locally, within the personal electronic device, such that a health condition report may be generated locally and delivered via a device user interface.

Sets of images associated with different patients may be stored within a network-connected database. New configurations for a pattern recognition module may be developed, to identify different patterns associated with different health conditions. Previously-stored image sets may then be analyzed for new patterns, to generate reports concerning new health conditions. Notifications of new health condition reports may be transmitted to one or more patients associated with analyzed sets of images. In some embodiments, new configurations for a pattern recognition module may be determined by also receiving health history information associated with one or more of the patients for whom scan images are stored. The server may calculate correlations between health history information associated with a patient, and patterns within the patient's scan images. New configuration patterns may then be developed based at least in part on the correlation calculations.

These and other methods and apparatuses will become apparent in light of the below information and associated drawings.

### BRIEF DESCRIPTION OF THE DRAWINGS

FIG. 1 is schematic block diagram of a system for health evaluation, in accordance with an exemplary embodiment.

FIG. 2 is a process for capturing and evaluating fingerprint scan data for identifying health conditions.

FIG. 3 is a process for identifying correlations between body scan data and health conditions.

### DETAILED DESCRIPTION OF THE DRAWINGS

While this invention is susceptible to embodiment in many different forms, there are shown in the drawings and will be described in detail herein several specific embodiments, with the understanding that the present disclosure is to be considered as an exemplification of the principles of the invention to enable any person skilled in the art to make and use the invention, and is not intended to limit the invention to the embodiments illustrated.

In accordance with some embodiments, a portable health evaluation tool may be implemented using relatively inexpensive personal electronic devices, including devices that many users may already own and use for other purposes. The tool scans measurable regions of a user's body, and compares those scans to a relational database in order to predict a present or future condition.

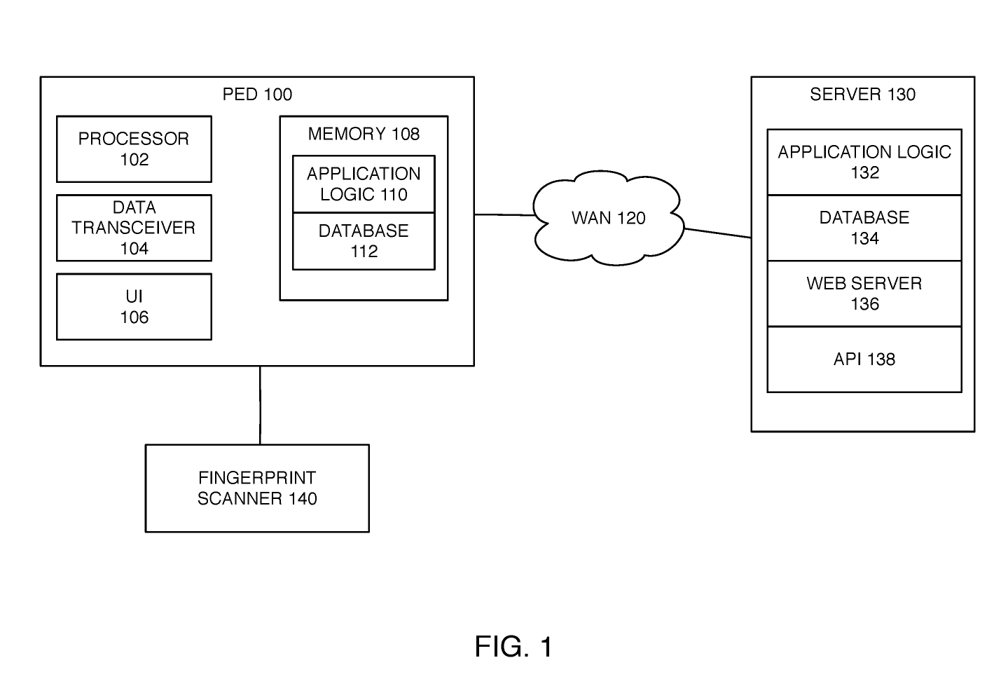

FIG. 1 illustrates an exemplary embodiment. Personal electronic device (PED) 100 communicates with server 120 via wide area network 110. In varying embodiments and use cases, PED 100 may be, without limitation: a smartphone, tablet computer, laptop computer, desktop computer, or smart watch. In a preferred embodiment, PED 100 is a smartphone implementing multiple wireless communication protocols, such as cellular data, wireless Ethernet and Bluetooth. PED 100 includes, inter alia, microprocessor 102, data transceiver 104, user interface components 106, and digital memory 108. Data transceiver 104 enables digital communications with other devices, and may include multiple network interfaces, such as a cellular modem, an Ethernet adapter, and a Bluetooth wireless transceiver. User interface components 106 enable a user of PED 100 to interact with the device; in various embodiments, UI 106 may include, without limitation, one or more of: a display screen, a touch-sensitive display screen, a keyboard, a mouse or trackpad, audio speakers, a microphone, and buttons. Digital memory 108 stores software and other data for processing by processor 102, including, without limitation: application logic 110 and database 112.

Wide area network (“WAN”) 120 may include one or more data networks interconnected to facilitate communications with, e.g., server 130. Typically, WAN 120 will include the Internet. In a use case in which PED 100 is a smartphone, WAN 120 may also include a wireless Ethernet local area network, a cellular data network, or both.

Server 130 is preferably an Internet-connected computation server implementing services for communication with one or more third party devices, such as web server 136 and Application Programming Interface (“API”) 138. Server 130 may also include database 134 and application logic 132. The term “database” is used herein broadly to refer to an indexed store of data, whether structured or not, including without limitation relational databases and document databases.

While personal electronic devices such as smartphones are increasingly ubiquitous avenues for computation and communication, health diagnostic applications for such devices typically require specialized hardware that may be costly and/or require significant expertise to operate. However, meaningful health diagnostic information may also be derived from analysis of scans of physical aspects of a user's body, which may be procured with little or no additional equipment, and little or no expertise in administration. In some embodiments, a user's skin prints may be analyzed. Skin prints refer to patterns formed by raised ridges typically found on some portions of the skin of an individual under examination. One useful type of skin print the fingerprint, formed by ridges on the tips of an individual's fingers.

Fingerprint and other skin print patterns may correlate with genetic expressions, which genetic expressions also correlate to certain health conditions, such as diseases and/or a user's propensity for developing certain diseases or other conditions. Moreover, skin print patterns may be procured by typical personal electronic devices with little additional equipment, and little expertise in operation.

In the embodiment of FIG. 1, fingerprint scanner 140 is provided. Fingerprint scanner 140 may be, for example, a fingerprint scanner having a wireless Bluetooth communications transceiver integrated into it for wireless communication to nearby PED 100. While scanner 140 may be referred to as a fingerprint scanner, it is contemplated and understood that in some embodiments, scanner 140 may be any skin imaging device capable of capturing a set of one or more digital images of a patient's skin patterns, such as palm prints, toe prints, or the like.

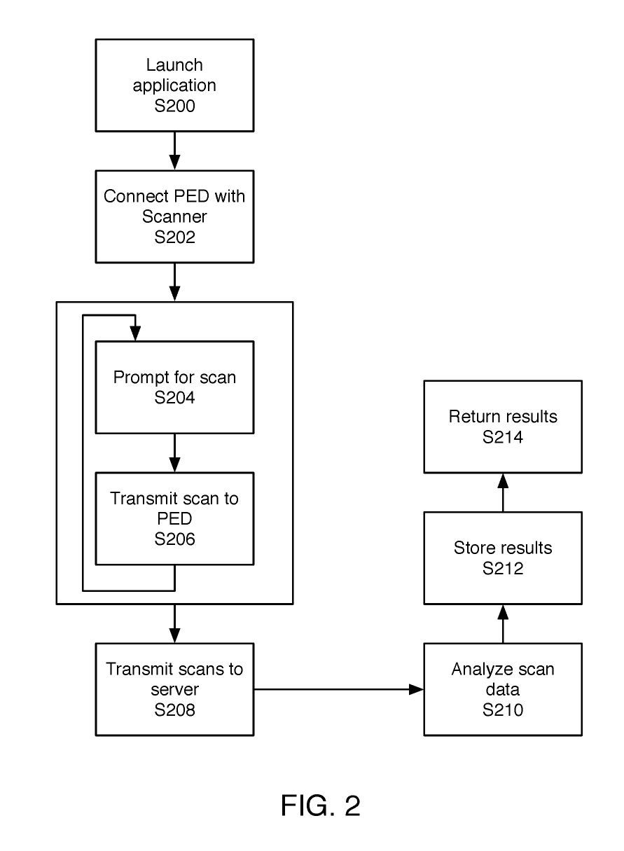

FIG. 2 is a process that may be implemented by the components of FIG. 1, in order to provide health diagnostic information to a user of PED 100. In step S200, a user of PED 100 launches an application stored in memory 108, via interaction with UI components 106. In step S202, PED 100 initiates wireless communications with fingerprint scanner 140. In step S204, PED 100 prompts the user to scan a fingerprint using scanner 140. In step S206, the scanned fingerprint is transmitted to PED 100 for storage within memory 108. In some embodiments, step S206 may also include a quality analysis, towards ensuring clarity of the scan and prompting the user to repeat the scan if quality is insufficient. In other embodiments, scan quality evaluation may be integrated within fingerprint scanner 140, such that the scanner 140 locally assesses scan quality and prompts the user to repeat scans deemed insufficient. Steps S204 and S206 are preferably repeated for each of the user's fingerprints, yielding a set of fingerprint images.

In step S208, the set of fingerprint images procured via scanner 140 is transmitted by PED 100 to server 130, for storage in database 134. In step S210, the fingerprint data is analyzed by server 130 for correlation with health conditions, to generate a report regarding a health condition of the patient. Any number of health conditions may be reported. In some embodiments, one health condition may be reported. In some embodiments, numerous health conditions may be reported. Health conditions may include the suspected presence or absence of a disease, or presence or absence of another type of health condition. Health conditions may also include a patient's likelihood of having and/or developing a particular disease or health condition, whether an absolute likelihood or likelihood relative to other individuals.

In step S212, results of step S210 are stored within server database 134. In step S214, a report based in whole or in part on the results of step S210 is returned to the user. In some embodiments, results may be returned by transmitting an analysis result notification message, such as an email, text message and/or app notification, to a user of PED 100 or someone associated therewith. The result notification may prompt the user to log into a web site implemented by web server 136, for viewing of analysis results. The result notification transmitted in step S214 may, in some circumstances, be directed to an individual other than those involved in procuring fingerprint scans in steps S204 and S206. For example, a nurse or medical assistant may assist in obtaining scans of a patient's fingers; however, server 130 may operate to transmit result notifications directly to a third party doctor having appropriate training and qualifications for proper interpretation of the results and conveyance thereof to the patient.

While results are preferably initially returned in step S214 to an individual with appropriate training and expertise to interpret the outcome and associated risk analysis, in some embodiments, results may be made available to a patient directly. In some embodiments, results may initially be accessible to a doctor via a doctor web portal implemented by web server 136. After communication of the test results to the patient, the doctor may then release results directly to the patient (e.g. via interaction with web server 136). In such embodiments, web server 136 may also implement a patient portal, via which a patient can log in to web server 136 and access test result reports and associated information directly.

While the embodiment of FIG. 1 illustrates use of a separate fingerprint scanner device 140, in other embodiments, other mechanisms for procuring fingerprint scans may be utilized. For example, many smartphones have built-in high-resolution cameras capable of focusing at short distances. In embodiments using such a device as PED 100, it may be desirable to enable an application operating on the device to photograph fingerprints imprinted on paper using an ink blotter, providing a low-cost and/or easily accessible alternative to fingerprint scanner 140. In such embodiments, steps S202 through S206 of the process of FIG. 2 may be replaced with user prompts on UI 106, to photograph previously-procured ink fingerprints. Appropriate image processing may be applied to such photographs, such as cropping of a fingerprint portion within a photograph frame, and adjusting levels and contrast to optimize fingerprint pattern recognition. In other circumstances, a personal electronic device may have a fingerprint scanner or other imaging device capable of capturing skin print patterns, built into it. To the extent that output from such a scanner is made available to applications running thereon, such an integrated scanner may also be utilized to procure scan data.

While fingerprints may be used in some use cases, in other use cases, embodiments may operate to scan portions of the body other than the finger. For example, toe prints, palm prints, and various combinations of prints from different parts of the body, may be desirable for analysis using the systems and methods described herein.

Various types of scan analysis may be performed in step S210. Different analyses may be effective in deriving different types of health information. For example, some types of health information may be associated with predetermined patterns (e.g. loops, whorls, ridges, and arches) appearing in particular locations on a user's digits and/or with particular frequency. A pattern recognition module implemented by application logic 132, executed by an onboard microprocessor within server 130, may operate on user scans to identify the presence and location of predetermined skin patterns and generate an output characterizing same. An evaluation module, also implemented by application logic 132 and an associated microprocessor, may then score a user's scans, comparing them to a relational database (within database 134) of patterns known to be associated with certain illnesses or other conditions.

One such analysis that may be particularly effective is evaluation of risk for ovarian cancer. Blood tests for ovarian cancer may not produce results, particularly early in a woman's life. Reliable tests for ovarian cancer often will not be effective until the cancer has progressed to a point that treatment may be ineffective in saving the patient's life. However, patterns revealed in an individual's fingerprint scans may be reflective of genetic sequences that cause ovarian cancer, such that embodiments described herein may be used to provide early warnings to individuals more likely to develop that disease. Various correlations between skin print patterns and health conditions, or likelihood of developing health conditions, may be determined by studies and analysis, and subsequently implemented by embodiments described herein via implementation of additional or different image analysis algorithms in step S210.

While the embodiment of FIG. 2 provides for scan analysis by server 130, in other embodiments, some or all scan analysis may be performed locally by PED 100. In such embodiments, application logic 110 includes image processing components for deriving health assessments from scan data. Deriving health assessments locally on PED 100 may mitigate certain data privacy concerns, to the extent that source scan data and health result data need not be transmitted or stored outside PED 100. On the other hand, in some embodiments it may be preferable to store confidential health data only on a central cloud server 130, which may implement appropriate encryption and data security measures to minimize risk of improper disclosure, thereby reducing data security issues presented by storage of confidential health information by numerous different individual doctor offices and other facilities maintaining and operating PEDs 100.

Some correlations of fingerprints or other scan patterns and health conditions, are known, and may be evaluated by directly processing a user's scans. However, other correlations of fingerprint or other scan patterns to health conditions, may be currently unknown, but available for discovery. Therefore, it may be desirable for server 130 to store scan data within database 134 for an extended period of time, along with patient profile information that includes an electronic communication address (such as email or SMS) associated with the individual. Then, as new techniques for identifying health conditions from scan data are developed, corresponding new configurations may be applied to a pattern recognition module within application logic 132, to implement new pattern recognition operations on previously-analyzed scan data, towards identifying responsive individuals. Previously-scanned individuals may then be notified of the results of those new diagnostic analyses, such as via email notification and/or reports made available via web server 136.

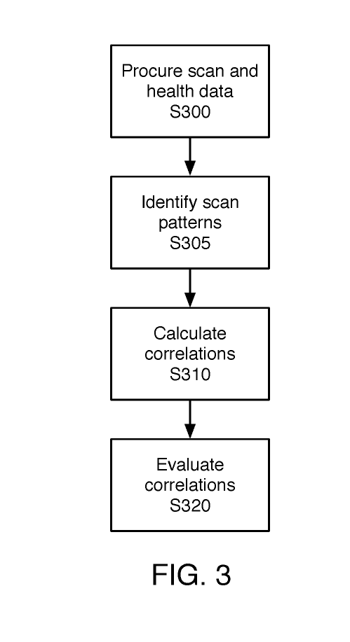

Scan data stored in database 134 may also be used for purposes of discovering new correlations with health conditions. FIG. 3 illustrates an exemplary process for such correlation discovery. In step S300, server 130 procures scan data and health condition data from numerous individuals. Scan data may be procured as described above, in connection with FIG. 2. Health condition data may be procured by a variety of techniques. For example, in some embodiments, PED 100 may present users with health questionnaires in order to obtain a health history of the patient. In other embodiments, server 130 may communication with systems providing access to patient electronic medical records, in order to ingest health condition information associated with patients from whom scans are received. In step S305, scan data is evaluated by server 130 to determine the presence or absence of one or more scan patterns. In step S310, server 130 calculates correlations between scan patterns identified in step S305, and associated health history data. In step S320, correlations are evaluated for diagnostic power; for example, correlations exceeding threshold parameters (e.g. high positive or high negative correlations) may be deemed of value, and potentially applied to user scan data both prospectively and retroactively, as described above.

While certain embodiments of the invention have been described herein in detail for purposes of clarity and understanding, the foregoing description and Figures merely explain and illustrate the present invention and the present invention is not limited thereto. It will be appreciated that those skilled in the art, having the present disclosure before them, will be able to make modifications and variations to that disclosed herein without departing from the scope of the invention or appended claims.

1. A method for evaluating a patient's health condition comprising:
capturing a set of one or more images of a patient's skin print patterns by a personal electronic device;
executing computer-implemented skin pattern recognition application logic to evaluate the set of images; and
based on output of the pattern recognition application logic, generating a report indicative of a health condition of the patient.

2. The method of claim 1, in which the step of capturing a set of images comprises:
imaging a patient's skin using a fingerprint scanner;
wirelessly transmitting a set of one or more images of the patient's skin print patterns from the fingerprint scanner to a personal electronic device; and
transmitting the set of images from the personal electronic device to a network-connected server.

3. The method of claim 1, in which the step of executing computer-implemented skin pattern application logic to evaluate the set of images comprises identifying a predetermined combination of patterns satisfying location and/or frequency criteria.

4. The method of claim 1, in which the step of generating a report indicative of a health condition of the patient comprises generating a report conveying relative likelihood of a patient having a predetermined health condition.

5. The method of claim 1, in which the step of capturing a set of one or more images comprises the substep of transmitting the set of one or more images to a network-connected server computer; and
in which the step of executing computer-implemented skin pattern recognition application logic is performed by the server computer.

6. The method of claim 5, further comprising the steps of:
storing the report within a network-connected database; and
publishing the report by a web server via a portal made available to the patient and/or a patient representative.

7. The method of claim 1, in which the step of executing computer-implemented skin pattern recognition application logic to evaluate the set of images is performed by the personal electronic device.

8. The method of claim 7, in which the step of generating a report indicative of a health condition of the patient is performed by the personal electronic device.

9. A health diagnostic apparatus comprising:
a skin print scanner capturing one or more images of a portion of a patient's skin;
a personal electronic device comprising a microprocessor in operable communication with the skin print scanner to receive patient skin print images therefrom;
computer-implemented analysis application logic configured to test for the presence of predetermined patterns within the skin print images

10. The apparatus of claim 9, in which the application logic comprises:
a pattern recognition module receiving said skin print images and outputting an evaluation of predetermined patterns identified therein; and
an evaluation module comparing the pattern recognition module output to one or more patterns associated with a health condition.

11. The apparatus of claim 9, in which the analysis application logic is implemented within a network-connected server communicating with the personal electronic device via the Internet.

12. The apparatus of claim 9, in which the skin print scanner is a standalone fingerprint scanner device comprising a wireless transceiver operable to transmit skin print images to the personal electronic device.

13. The apparatus of claim 9, in which the skin print scanner is integrated within the personal electronic device.

14. A method for evaluating a patient's health condition comprising:
receiving from one or more personal electronic devices, by a network-connected server, multiple sets of one or more images of patient skin print patterns, each set associated with a patient;
storing said multiple sets of images of skin print patterns within a database;
subsequently applying a new configuration to a pattern recognition module implemented by the server; and
executing the pattern recognition module new configuration against the previously-stored sets of images within the database to generate new health condition reports for patients associated with the sets of images.

15. The method of claim 14, further comprising the step of transmitting notifications of new health condition reports to one or more of said patients.

16. The method of claim 13, in which:
the step of receiving multiple sets of one or more images of patient skin print patterns, each set associated with a patient, further comprises the step of receiving a record of health history information associated with one or more of said patients; and
the step of subsequently applying a new configuration to a pattern recognition module implemented by the server comprises: calculating correlations between health history information associated with a patient and patterns identified within a set of images of skin print patterns associated with the same patient; and developing a new configuration for a pattern recognition module based at least in part on the correlation calculations.
